(19) United States
(12) Patent Application Publication
(10) Pub. No.: US 2007/0292414 A1
(43) Pub. Date: Dec. 20, 2007

(54) Compositions enriched in neoplastic stem cells and methods comprising same

(76) Inventors: Christopher Duntsch, Memphis, TN (US); Valery Kukekeov, Memphis, TN (US); Tatyana Igantova, Memphis, TN (US)

Correspondence Address: PEARL COHEN ZEDEK LATZER, LLP, 1500 BROADWAY 12TH FLOOR, NEW YORK, NY, 10036, US

(21) Appl. No.: 11/806,993

(22) Filed: Jun. 5, 2007

Related U.S. Application Data
(60) Provisional application No. 60/811,095, filed on Jun. 6, 2006.

(51) Int. Cl. A61K 38/00; A01N 63/00; A61K 39/395; A61P 37/00; C12N 5/02; C12N 5/04; C12Q 1/02; C12Q 1/68

(52) U.S. Cl. 424/130.1; 424/93.7; 435/29; 435/325; 435/378; 435/383; 435/6; 514/44

(57) ABSTRACT

A neoplastic stem cell population enriched for expression of the OCT4 transcription factor as well as methods for their identification, isolation and enrichment are described. The OCT4-enriched neoplastic stem cell population is further utilized for the induction and analysis of cancer in an animal. In addition, methods of preventing, abrogating, or inhibiting cancer, tumor growth, and metastasis via OCT4 inhibition are further provided.

### CROSS REFERENCE TO RELATED APPLICATIONS

This application claims the benefit of U.S. Application Ser. No. 60/811,095, filed on Jun. 6, 2006, which is incorporated in its entirety herein by reference.

### BACKGROUND OF THE INVENTION

To date, methods of analysis of neoplastic cells are neither efficient nor uniform enough for research purposes. Neoplastic stem cells isolated from tissue samples by various fractionation procedures consisted of mixed cell types. Efficient isolation of neoplastic stem cells provides a means of exploring basic mechanisms in cancer cell biology and disease. Methods for specifically and efficiently isolating and propagating a cell subpopulation to provide a large neoplastic stem cell population for in-vitro and in-vivo studies are desirable.

Identification of stem cell markers in neoplastic cells provides valuable information that is useful for a variety of applications in both clinical and basic research settings. The identification and subsequent isolation of neoplastic stem cells (NSCs) from a particular tumor or metastatic lesion is useful, for example, in diagnosing a pathology and/or developing a rational therapeutic treatment that targets a developing pathology. In some instances, isolation and/or enrichment of NSCs is desirable for further in-vitro studies exploring physiological and molecular mechanisms, wherein in other instances, these cells can be used to inoculate a test animal for further studies of cancer progression or therapy.

NSCs can be sorted using techniques such as FACS (FIG. 10) or antibiotic selection assays that do not distinguish between sub-populations of cells based on their biological activity and/or physiological function. The assays, moreover, preclude recovery of native non-antibiotic-expressing or treated stem cells. Other methods of cellular identification and subsequent isolation and/or enrichment such as gel electrophoresis, fail to probe pure populations, suffer from contamination and/or compromise cell viability.

### SUMMARY OF THE INVENTION

This invention relates, in another embodiment, to a neoplastic stem cell population enriched for expression of OCT4.

In another embodiment, this invention provides a method of identifying neoplastic stem cells, comprising the steps of (i) contacting neoplastic cells with an agent which specifically interacts with OCT4; and (ii) identifying cells with which the agent specifically interacts.

In another embodiment, this invention provides a method of isolating neoplastic stem cells, comprising the steps of (i) contacting neoplastic cells with an agent which specifically interacts with OCT4; and (ii) isolating cells with which the agent specifically interacted.

In another embodiment, this invention provides a method of enriching a neoplastic cell population for neoplastic stem cells, comprising the steps of (i) contacting a mixed cell population comprising a plurality of cancerous cells with a vector comprising an antibiotic resistance gene operatively linked to an Oct4 promoter; and (ii) culturing the mixed cell population in the presence of an antibiotic.

In another embodiment, this invention provides a method of inducing cancer comprising introducing a neoplastic stem cell population enriched for expression of OCT4 to a mammal.

In another embodiment, this invention provides a method of analyzing cancer progression and/or pathogenesis in-vivo, comprising the steps of (i) transplanting OCT4^{hi} neoplastic stem cells into an animal; and (ii) analyzing cancer progression and/or pathogenesis in the animal.

In another embodiment, this invention provides a method of abrogating, or inhibiting cancer comprising the step of: contacting neoplastic cells with an agent that inhibits OCT4 expression or function in said neoplastic cells.

In another embodiment, this invention provides a method of preventing, abrogating, or inhibiting tumor growth comprising the step of: contacting neoplastic cells with an agent that inhibits OCT4 expression or function in a tumor.

In another embodiment, this invention provides a method of preventing, abrogating, or inhibiting cell metastasis comprising the step of: contacting neoplastic cells with an agent that inhibits OCT4 expression or function in said neoplastic cells.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 presents light micrographs of serial sections of solid tumors, probed with polyclonal anti-OCT4 antibodies and control sections. FIG. 1A is a cross section of a chondrosarcoma tumor; FIG. 1B is a cross section of an osteosarcoma tumor; FIG. 1C is a cross section of a glioblastoma multiforme (GBM) tumor; and FIG. 1D is a cross section of fetal human testis used for positive control. Arrows indicate OCT4 positive nuclei.

FIG. 2A demonstrates the results of semi-quantitive RT-PCR probing for OCT4, STAT3 and Nanog mRNA expression wherein β-tubulin and GFAP served as normalized controls in representative glioblastoma primary cultures (MT917, MT926, MT928, MT1231) and cell lines (LN18, LN229, LN428, U251). FIG. 2B demonstrates results of a Western blot analysis of OCT4, STAT3 and Nanog protein expression wherein β-tubulin and GFAP served as normalized controls in cell lines (LN18, LN229, LN319, LN428, D247, U251, U373, T98G). Blots were probed with anti-OCT4, anti-phospho-STAT3 and anti-Nanog.

FIG. 3 plots the results of 2-D quantitative PCR probing OCT4 and nanog gene expression in adherent cell cultures and floating osteosarcoma-derived spheres. Substrate-attached cultures showed significantly (p<0.05) lower expression of OCT4 and Nanog. Correlation of OCT4 (X axis) and Nanog (Y axis) expression in sarcospheres is significantly (p<0.05) higher than in substrate-attached cultures.

FIG. 4 illustrates clone-forming potential of glioblastoma-derived cells suppressed by OCT4 siRNA. FIG. 4A demonstrates results of a Western blot analysis wherein suppression of exogenous OCT4 protein in a transfected cell culture was achieved by treatment with specific OCT4 siRNA comprising the DNA sequence: TTGATCCTCGGACCTGGCTAA. FIG. 4B plots the frequency of clone-formation by selected glioblastoma cells (MT317, LN-229, MT-917). Cells were co-transfected with eGFP (Green Fluorescent Protein) and OCT4 siRNA. Experiments were performed in triplicate, bars represent standard errors.

FIG. 5A is a light micrograph image (×200) of suspended mammaspheres derived from an MCF-7R breast cancer cell line and cultured in methylcellulose. FIG. 5B is a fluorescent micrograph (×200) of a mammasphere transferred from methylcellulose, attached to substratum and immunostained for OCT4 (white) and pancytokeratine (gray) expression.

FIG. 6 presents light micrographs (×200) of immunohistochemically stained breast cancer tumors probed for OCT4 expression. Dark punctuate staining are OCT4 positive nuclei. FIG. 6A is a cross section of ductal carcinoma tumor, and FIG. 6B is a cross section of breast cancer metastasis to brain. Arrows indicate OCT4 positive nuclei.

FIG. 7 is a fluorescent microscope (×200) image of an OCT4-EGFP transfected glioblastoma cell in methyl cellulose after first division (FIG. 7A). A glioblastoma floating neurosphere (clone) of OCT-EGFP transfected cells after several rounds of divisions is shown in FIG. 7B.

FIG. 8 is a fluorescent microscope image (×200) of cultured breast cancer cells (A), osteosarcoma (B), and glioblastoma multiforme cells (C) expressing EGFP through an OCT4 responsive promoter.

FIG. 9 is a fluorescent microscope image of the cultured glioblastoma cell line Ln428 (×200) (A) and osteosarcoma OS521 (×100) (B) expressing EGFP through Nanog responsive promoter.

FIG. 10 illustrates FACS flow isolation graphs of subpopulations of tumor cells expressing OCT4 from cultured glioblastoma cell line Ln428. M2 gate represents OCT4 positive cells. FIG. 10B represents the initial FACS sorting for OCT4 positive cells of a mixed clonal-OCT4 cell population. FIG. 10B represents isolated OCT4 positive cells after two passages (roughly 2 weeks) followed by FACS analysis (B) to determine their purity. This population was found to be 96.16% pure for OCT4 protein expression.

FIG. 11 is a graph illustrating the tumor forming potential of OCT4 positive and OCT4 negative MDA MB 231 breast cancer cells transfected with OCT4-EGFR.

FIG. 12 schematically depicts the procedure for obtaining OCT4 enriched tumor stem cells from any tumor tissue for cancer related studies including drug discovery studies. Stage A: Preparation of Cells: 1) Surgical removal of tumor 2) Mincing and preparations to create a single cells suspension. Stage B: Stable labeling of tumor stem cells with OCT4 responsive promoter: 1) Tumor stem cell culture in a single cell suspension for expansion and selection for tumor stem cells under the appropriate conditions 2) transfection with a plasmid comprising an EGFP gene under the control of an OCT4 responsive promoter (stage C). Stage C: Creation of a highly pure tumor stem cell cultures: EGFP expressing cells are further selected via FACS and re-cultured for expansion resulting in bulk culture quantities. Stage D: D1, tumor stem cells are further studied using rigorous cell and molecular biology techniques. D2, tumor stem cells are exposed to a vast variety of drugs Stage E: Isolated tumor stem cells are inoculated into immunodeficient mice to create xenograft tumor models followed by basic efficacy, safety (or lack of toxicity), and outcomes studies generating final drug lists.

FIG. 13 is a microscope image (×200) of mammosphere cultures derived from an MDA-MB-435 melanoma cell line, biomarked for the presence of NSC expressing Oct-3/4. FIG. 13A is a light micrograph of suspended tumor-derived spheres cultured in methylcellulose. FIG. 13B is a fluorescent micrograph of the suspended tumor-derived spheres shown in FIG. 13A. FIG. 13C is a light micrograph of tumor spheres after attachment to the substratum. FIG. 13D is a fluorescent micrograph of the attached tumor spheres shown in FIG. 13C.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

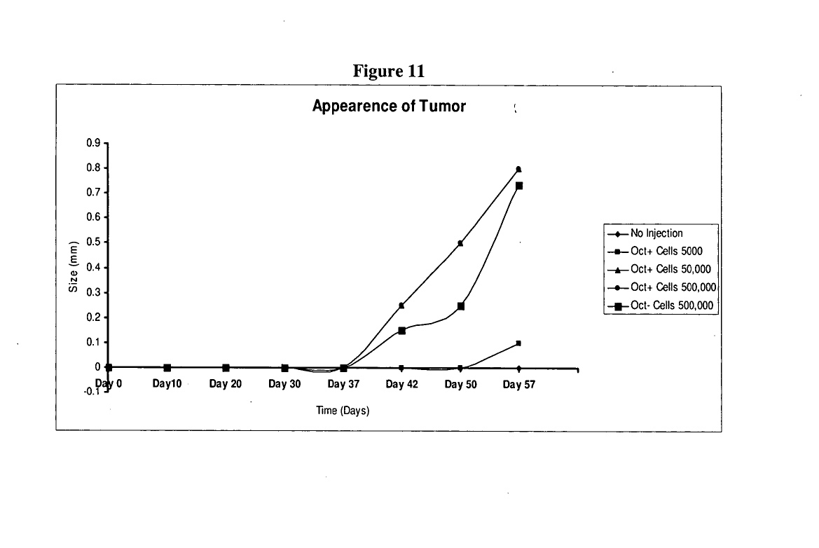

In another embodiment, the invention comprises a neoplastic stem cell (NSC) population enriched for expression of OCT4, Nanog, STAT3 or combinations thereof. In another embodiment, NSCs represent a subpopulation of cells within a population comprising neoplastic cells, which is capable of initiating and maintaining cancer following a prolonged period of time. In another embodiment, NSCs drive the formation and growth of tumors (FIG. 11). In another embodiment, the term drive as used herein refers to guide, control, direct, initiate, go through, penetrate or combinations thereof.

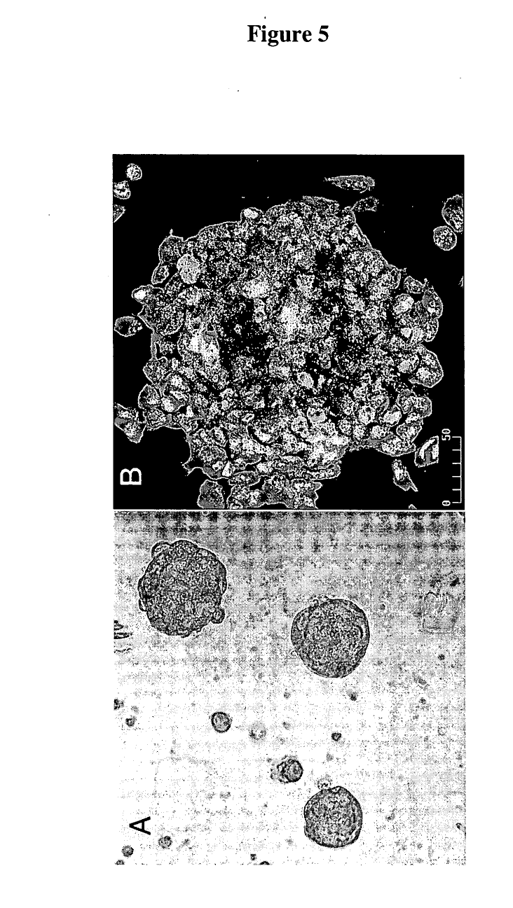

In another embodiment, NSCs comprise properties such as longevity, self-renewal and quiescence. In another embodiment, NSCs comprise enhanced invasive capacity. In another embodiment, NSCs are multipotent, self-renewing and are able to produce proliferating sarcospheres from sarcomas, neurospheres from brain tumors or mammaspheres from breast cancers (FIG. 5). In another embodiment, NSCs are capable of keeping their self-renewal potential during 1-100 passages of in-vitro cultivation. In another embodiment, NSCs are capable of keeping their self-renewal potential during 1-90 passages of in-vitro cultivation. In another embodiment, NSCs are capable of keeping their self-renewal potential during 20-60 passages of in-vitro cultivation. In another embodiment, NSCs express genes involved in the specific functions and/or in self-renewal of NSCs, such as OCT4, Nanog, STAT3 or combinations thereof.

In another embodiment, the present invention provides that solid cancer represents a population of cells derived from a common founder cell, or NSC. In another embodiment, the present invention provides that tumors represent a population of cells derived from a common founder cell, or NSC. In another embodiment, the present invention provides that NSC phenotype is similar in many ways to that of normal stem cells. In another embodiment, the present invention provides that NSC phenotype is quite different to that of normal stem cells leading to the irregularities with respect to abnormal developmental profile. In another embodiment, the present invention provides that NSC phenotype is quite different from that of normal stem cells leading to the irregularities with respect to lack of key proliferation controls.

In another embodiment, the present invention provides that NSC population comprises a mix of true or mother NSCs and the progenitors neoplastic cells derived from NSCs. In another embodiment, the present invention provides that progenitors derived from NSCs are different in key ways from mother NSCs. In another embodiment, the present invention provides that different in key ways comprise high proliferation kinetics. In another embodiment, the present invention provides that NSCs are typically present in very low percentages relative to the total cancer cell population, correlating roughly to the “hostility” of the environment (i.e., a natural environment such as a breast NSC in its primary breast tissue location versus a breast NSC located in a metastatic and/or foreign location such as the brain.

In another embodiment, the present invention provides that NSCs comprise about 0.001 to 1% of the parental primary cancer population. In another embodiment, the present invention provides that NSCs comprise about 0.005 to 1% of the parental primary cancer population. In another embodiment, the present invention provides that NSCs comprise about 0.01 to 0.1% of the parental primary cancer population. In another embodiment, the present invention provides that NSCs comprise about 0.05 to 0.1% of the parental primary cancer population. In another embodiment, the present invention provides that NSCs comprise about 0.005 to 0.01% of the parental primary cancer population.

In another embodiment, the present invention provides that NSCs comprise about 1 to 80% of the cell population in permanent cancer cell lines parental. In another embodiment, the present invention provides that NSCs comprise about 1 to 10% of the cell population in permanent cancer cell lines parental. In another embodiment, the present invention provides that NSCs comprise about 7 to 14% of the cell population in permanent cancer cell lines parental. In another embodiment, the present invention provides that NSCs comprise about 15 to 25% of the cell population in permanent cancer cell lines parental. In another embodiment, the present invention provides that NSCs comprise about 10 to 30% of the cell population in permanent cancer cell lines parental. In another embodiment, the present invention provides that NSCs comprise about 30 to 50% of the cell population in permanent cancer cell lines parental. In another embodiment, the present invention provides that NSCs comprise about 20 to 40% of the cell population in permanent cancer cell lines parental. In another embodiment, the present invention provides that NSCs comprise about 50 to 80% of the cell population in permanent cancer cell lines parental. In another embodiment, the present invention provides that NSCs comprise about 1 to 5% of the cell population in permanent cancer cell lines parental. In another embodiment, the present invention provides that NSCs comprise about 5 to 10% of the cell population in permanent cancer cell lines parental. In another embodiment, the present invention provides that NSCs comprise about 3 to 8% of the cell population in permanent cancer cell lines parental. In another embodiment, the present invention provides that NSCs comprise about 7 to 10% of the cell population in permanent cancer cell lines parental.

In another embodiment, the present invention provides that NSCs comprise about 1 to 100% of the parental metastatic cancer population cell. In another embodiment, the present invention provides that NSCs comprise about 1 to 10% of the parental metastatic cancer population cell. In another embodiment, the present invention provides that NSCs comprise about 10 to 30% of the parental metastatic cancer population cell. In another embodiment, the present invention provides that NSCs comprise about 30 to 50% of the parental metastatic cancer population cell. In another embodiment, the present invention provides that NSCs comprise about 50 to 75% of the parental metastatic cancer population cell. In another embodiment, the present invention provides that NSCs comprise about 75 to 100% of the parental metastatic cancer population cell. In another embodiment, the present invention provides that NSCs comprise about 30 to 80% of the parental metastatic cancer population cell. In another embodiment, the present invention provides that NSCs comprise about 20 to 90% of the parental metastatic cancer population cell. In another embodiment, the present invention provides that NSCs comprise about 10 to 100% of the parental metastatic cancer population cell. In another embodiment, the present invention provides that NSCs comprise about 20 to 40% of the parental metastatic cancer population cell.

In another embodiment, the present invention provides that bulk cancer cells (BCCs) comprise the majority of the cancer cell population from a primary solid tumor. In another embodiment, the present invention provides that BCCs comprise the majority of the cancer cell population from a permanent cultured cell lines derived from cancers. In another embodiment, the present invention provides that a BCC population lacks stem cell characteristics. In another embodiment, the present invention provides that a BCC population lacks OCT-4 expression.

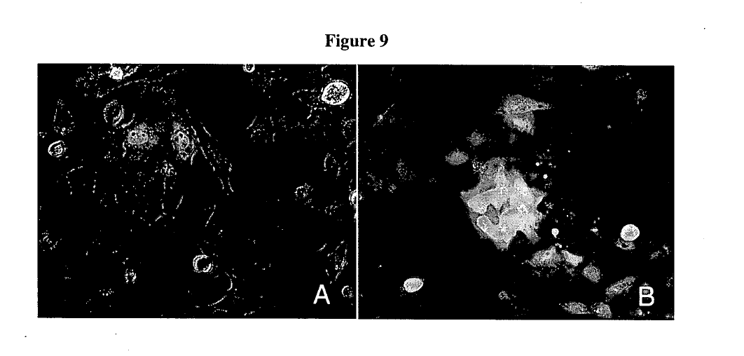

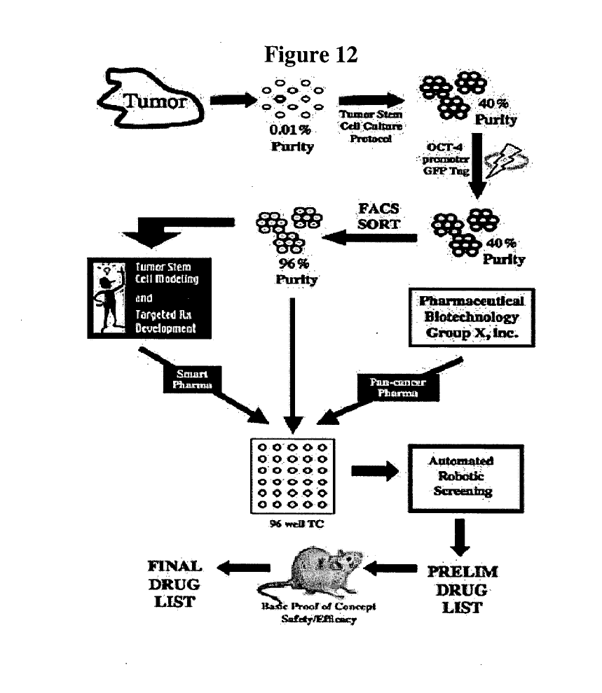

In another embodiment, the methods of the present invention provides isolation of NSCs from cancer tissue biopsies and permanent cancer cell lines by selection of NSCs previously manipulated and biomarked to allow for detection. In another embodiment, the methods of the present invention provide stably transfecting NSCs with DNA vectors which expresses fluorescent or luminescent proteins regulated by an Oct-4 responsive promoter (FIG. 12). In another embodiment, the methods of the present invention provides separating NSCs from the total cancer cell population resulting in cultures of high purity using FACS sorting of fluorescent biomarkers. In another embodiment, the methods of the present invention provides separating NSCs from the total cancer cell population resulting in cultures of high purity using FACS sorting of those cells that express GFP (Green Fluorescent Protein) driven by an Oct4 promoter (FIG. 9).

In another embodiment, the sequence of the Oct-4 cDNA of the present invention comprises the sequence: tcccttcgcaagccctcatttcaccaggccccggcttggggcgccttccttccccatggcgggacacctggcttcggatttcgccttctcgcc cctccaggtggtggaggtgatgggccaggggggccggagccgggctgggttgatcctcggacctggctaagcttccaaggccctcctggagggccag gaatcgggcgggggttgggccaggctctgaggtgtgggggattcccccatgccccccgccgtatgagttctgtggggggatggcgtactgtgggccc caggttggagtggggctagtgcccaaggcggcttggagacctctcagcctgagggcgaagcaggagtcggggtggagagcaactccgatggggcc tccccggagccctgcaccgtcacccctggtgccgtgaagctggagaaggagaagctggagcaaaacccggaggagtcccaggacatcaaagctctgc agaaagaactcgagcaatttgccaagctcctgaagcagaagaggatcaccctgggatatacacaggccgatgtggggctcaccctgggggttctatttgg gaaggtattcagccaaacgaccatctgccgctttgaggctctgcagcttagcttcaagaacatgtgtaagctgcggcccttgctgcagaagtgggtggagg aagctgacaacaatgaaaatcttcaggagatatgcaaagcagaaaccctcgtgcaggcccgaaagagaaagcgaaccagtatcgagaaccgagtgag aggcaacctggagaatttgttcctgcagtgcccgaaacccacactgcagcagatcagccacatcgcccagcagcttgggctcgagaaggatgtggtccg agtgtggttctgtaaccggcgccagaagggcaagcgatcaagcagcgactatgcacaacgagaggattttgaggctgctgggtctcctttctcaggggga ccagtgtcctttcctctggcccccagggccccattttggtaccccaggctatgggagccctcacttcactgcactgtactcCtcggtccctttccctgaggggg aagcctttccccctgtctccgtcaccactctgggctctcccatgcattcaaactgaggtgcctgccttctaggaatgggggacagggggaggggaggag ctagggaaagaaaacctggagtttgtgccagggtttttgggattaagttcttcattcactaaggaaggaattgggaacacaaagggtgggggcaggggag tttggggcaactggttggagggaaggtgaagttcaatgatgctcttgattttaatcccacatcatgtatcacttttttcttaaataaagaagcctgggacacagt aaaaaaaaaaaaaaaaaaaaaaaaaaaaa (SEQ. ID NO: 1). In another embodiment, the Oct-4 CDNA the present invention comprises a nucleic acid sequence homologous to SEQ. ID. NO: 1. In another embodiment, the Oct-4 CDNA sequence is a Homo sapiens Oct-4 CDNA sequence. In another embodiment, the Oct-4 CDNA sequence is from a non-human species. Each possibility represents a separate embodiment of the present invention.

In another embodiment, the sequence of the Oct-4 CDNA of the present invention comprises the sequence: gtagtcctttgttacatgcatgagtcagtgaacagggaatgggtgaatgacatttgtgggtaggttatttctagaagttaggtgggcagcttgg aaggcagaggcacttctacagactattccttggggccacacgtaggttcttgaatcccgaatggaaaggggagattgataactggtgtgtttatgttcttaca agtcttctgccttttaaaatccagtcccaggacatcaaagctctgcagaaagaactcgagcaatttgccaagctcctgaagcagaagaggatcaccctggg atatacacaggccgatgtggggctcaccctgggggttctatttgggaaggtattcagccaaacgaccatctgccgctttgaggctctgcagcttagcttcaa gaacatgtgtaagctgcggccttgctgcagaagtgggtggaggaagctgacaacaatgaaaatcttcaggagatatgcaaagcagaaaccctcgtgca ggcccgaaagagaaagcgaaccagtatcgagaaccgagtgagaggcaacctggagaatttgttcctgcagtgcccgaaacccacactgcagcagatc agccacatcgcccagcagcttgggctcgagaaggatgtggtccgagtgtggttctgtaaccggcgccagaagggcaagcgatcaagcagcgactatgc acaacgagaggattttgaggctgctgggtctcctttctcagggggaccagtgtcctttcctctggccccagggccccattttggtaccccaggctatgggag ccctcacttcactgcactgtactcctcggtccctttccctgagggggaagcctttcccctgtctccgtcaccactctgggctctcccatgcattcaaactgag gtgcctgcccttctaggaatgggggacagggggaggggaggagctagggaaagaaaacctggagtttgtgccagggtttttgggattaagttcttcattca ctaaggaaggaattgggaacacaaagggtgggggcaggggagtttggggcaactggttggagggaaggtgaagttcaatgatgctcttgattttaatccc acatcatgtatcacttttttcttaaataaagaagcctgggacacagtaaaaaaaaaaaaaaaaaaaaaaaaaaaaa (SEQ. ID NO: 2). In another embodiment, the Oct-4 CDNA the present invention comprises a nucleic acid sequence homologous to SEQ. ID. NO: 2. In another embodiment, the Oct-4 CDNA sequence is a Homo sapiens Oct-4 CDNA sequence. In another embodiment, the Oct-4 CDNA sequence is from a non-human species. Each possibility represents a separate embodiment of the present invention.

In another embodiment, the sequence of the OCT-4 protein of the present invention comprises the sequence: MAGHLASDFAFSPPPGGGGDGPGGPEPGWVDPRTWLSFQGPPGGPGIGPGVGPG SEVWGIPPCPPPYEFCGGMAYCGPQVGVGLVPQGGLETSQPEGEAGVGVESNSDGASPEPCTVT PGAVKLEKEKLEQNPEESQDIKALQKELEQFAKLLKQKRITLGYTQADVGLTLGVLFGKVFSQT TICRFEALQLSFKNMCKLRPLLQKWVEEADNNENLQEICKAETLVQARKRKRTSIENRVRGNLE NLFLQCPKPTLQQISHIAQQLGLEKDVVRVWFCNRRQKGKRSSSDYAQREDFEAAGSPFSGGPV SFPLAPGPHFGTPGYGSPHFTFALYSSVPFPEGEAFPPVSVTTLGSPMHSN (SEQ. ID NO: 3). In another embodiment, the OCT-4 protein of the present invention comprises an amino acid sequence homologous to SEQ. ID. NO: 3. In another embodiment, the OCT-4 protein is a Homo sapiens OCT-4 protein. In another embodiment, the OCT-4 protein is from a non-human species. Each possibility represents a separate embodiment of the present invention.

In another embodiment, the sequence of the OCT-4 protein of the present invention comprises the sequence: MCKLRPLLQKWVEEADNNENLQEICKAETLVQARKRKRTSIENRVRGNLENLFLQCPKPT LQQISHIAQQLGLEKDVVRVWFCNRRQKGKRSSSDYAQREDFEAAGSPFSGGPVSFPLAPGPHF GTPGYGSPHFTALYSSVPFPEGEAFPPVSVTTLGSPMHSN (SEQ. ID NO: 4). In another embodiment, the OCT-4 protein of the present invention comprises an amino acid sequence homologous to SEQ. ID. NO: 4. In another embodiment, the OCT-4 protein is a Homo sapiens OCT-4 protein. In another embodiment, the OCT-4 protein is from a non-human species. Each possibility represents a separate embodiment of the present invention.

n another embodiment, the sequence of the Oct-4 responsive promoter of the present invention comprises the cacccaggggcggggccagaggtcaaggctagagggtggg (SEQ. ID NO: 5). In another embodiment, the Oct-4 responsive promoter of the present invention comprises a nucleic acid sequence homologous to SEQ. ID. NO: 5. In another embodiment, the Oct-4 responsive promoter sequence is a murine Oct-4 responsive promoter sequence. In another embodiment, the Oct-4 responsive promoter sequence is from a Homo-sapiens. In another embodiment, the Oct-4 responsive promoter sequence is from a non-human species. Each possibility represents a separate embodiment of the present invention.

In another embodiment, the Oct-4 DNA sequence of the present invention is at least 60% homologous to anyone SEQ. ID NOs: 1-2. In another embodiment, the Oct-4 DNA sequence of the present invention is at least 70% homologous to anyone SEQ. ID NOs: 1-2. In another embodiment, the Oct-4 DNA sequence of the present invention is at least 80% homologous to anyone SEQ. ID NOs: 1-2. In another embodiment, the Oct-4 DNA sequence of the present invention is at least 90% homologous to anyone SEQ. ID NOs: 1-2. In another embodiment, the Oct-4 DNA sequence of the present invention is at least 95% homologous to anyone SEQ. ID NOs: 1-2.

In another embodiment, the Oct-4 responsive promoter DNA sequence of the present invention is at least 60% homologous to anyone SEQ. ID NOs: 5. In another embodiment, the Oct-4 responsive promoter DNA sequence of the present invention is at least 70% homologous to anyone SEQ. ID NOs: 5. In another embodiment, the Oct-4 responsive promoter DNA sequence of the present invention is at least 80% homologous to anyone SEQ. ID NOs: 5. In another embodiment, the Oct-4 responsive promoter DNA sequence of the present invention is at least 90% homologous to anyone SEQ. ID NOs: 5. In another embodiment, the Oct-4 responsive promoter DNA sequence of the present invention is at least 95% homologous to anyone SEQ. ID NOs: 5.

In another embodiment, the Oct-4 protein sequence of the present invention is at least 60% homologous to anyone SEQ. ID NOs: 3-4. In another embodiment, the Oct-4 protein sequence of the present invention is at least 70% homologous to anyone SEQ. ID NOs: 3-4. In another embodiment, the Oct-4 protein sequence of the present invention is at least 80% homologous to anyone SEQ. ID NOs: 3-4. In another embodiment, the Oct-4 protein sequence of the present invention is at least 90% homologous to anyone SEQ. ID NOs: 3-4. In another embodiment, the Oct-4 protein sequence of the present invention is at least 95% homologous to anyone SEQ. ID NOs: 3-4.

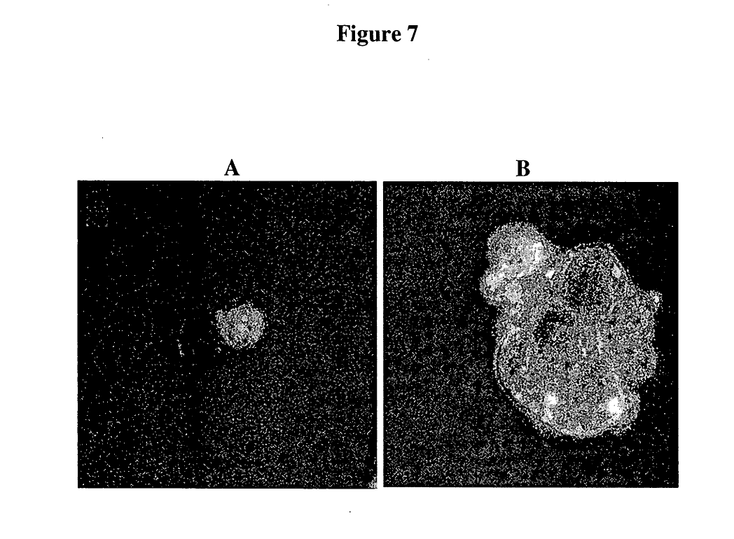

In another embodiment, the methods of the present invention provide a highly pure biomarked NSC population. In another embodiment, the methods of the present invention provides that a highly pure biomarked NSC population is studied in numerous ways by taking advantage of their fluorescent properties (FIGS. 7-9).

In another embodiment, the methods of the present invention provide that NSCs can be passaged without loosing their NSC phenotype for at least 5 passages. In another embodiment, the methods of the present invention provide that NSCs can be passaged without loosing their NSC phenotype for at least 8 passages. In another embodiment, the methods of the present invention provide that NSCs can be passaged without loosing their NSC phenotype for at least 10 passages. In another embodiment, the methods of the present invention provide that NSCs can be passaged without loosing their NSC phenotype for at least 15 passages. In another embodiment, the methods of the present invention provide that NSCs can be passaged without loosing their NSC phenotype for at least 20 passages. In another embodiment, the methods of the present invention provide that NSCs can be passaged without loosing their NSC phenotype for at least 25 passages. In another embodiment, the methods of the present invention provide that NSCs can be passaged without loosing their NSC phenotype for at least 30 passages. In another embodiment, the methods of the present invention provide that NSCs can be passaged without loosing their NSC phenotype for at least 35 passages. In another embodiment, the methods of the present invention provide that NSCs can be passaged without loosing their NSC phenotype for at least 40 passages. In another embodiment, the methods of the present invention provide that NSCs can be passaged without loosing their NSC phenotype for at least 45 passages. In another embodiment, the methods of the present invention provide that NSCs can be passaged without loosing their NSC phenotype for at least 50 passages.

In another embodiment, the methods of the present invention provide that NSCs can be passaged and retain Oct-4 expression for at least 5 passages. In another embodiment, the methods of the present invention provide that NSCs can be passaged and retain Oct-4 expression for at least 10 passages.

In another embodiment, the methods of the present invention provide that NSCs can be passaged and retain Oct-4 expression for at least 15 passages. In another embodiment, the methods of the present invention provide that NSCs can be passaged and retain Oct-4 expression for at least 20 passages. In another embodiment, the methods of the present invention provide that NSCs can be passaged and retain Oct-4 expression for at least 25 passages. In another embodiment, the methods of the present invention provide that NSCs can be passaged and retain Oct-4 expression for at least 30 passages. In another embodiment, the methods of the present invention provide that NSCs can be passaged and retain Oct-4 expression for at least 35 passages. In another embodiment, the methods of the present invention provide that NSCs can be passaged and retain Oct-4 expression for at least 40 passages. In another embodiment, the methods of the present invention provide that NSCs can be passaged and retain Oct-4 expression for at least 45 passages. In another embodiment, the methods of the present invention provide that NSCs can be passaged and retain Oct-4 expression for at least 50 passages.

In another embodiment, the methods of the present invention provide that NSC populations are expanded into large volume mass cultures for extended periods of time without losing their desired pure NSC phenotype. In another embodiment, the methods of the present invention provide that NSC populations are expanded into large volume mass cultures for extended periods of time without losing their Oct-4 expression.

In another embodiment, NSCs are enriched for a stem cell marker. In another embodiment, the stem cell marker is OCT4, Nanog, STAT3 or combinations thereof. In another embodiment, the stem cell marker is a transcription factor such as OCT4. In another embodiment, OCT4 is differentially expressed in NSCs. In another embodiment, immunological methods of enriching for OCT4 expressing cells based on their affinity to surface antigens are used. In another embodiment, NSCs are enriched by an immunomagnetic based cell separation technique. In another embodiment, NSCs are enriched by the electrophoretic cell separation technique based on the electrophoretic mobility reduction via incubation with antibodies specific to surface antigen. In another embodiment, the reduction in electrophoretic mobility by incubation with surface antigen specific antibodies is performed under non-capping conditions. In another embodiment, NSCs are further enriched through fluorescence-activated cell sorter (FACS), immunomagnetic beads, or magnetic-activated cell sorter (MACS).

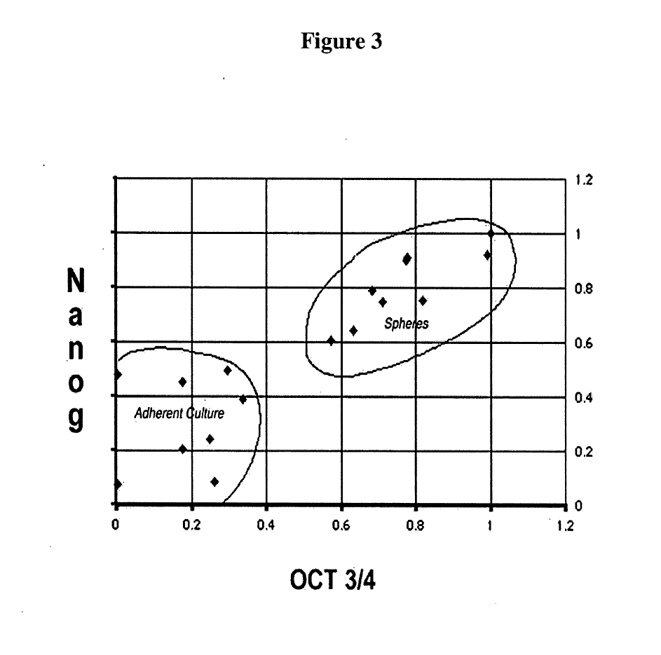

In another embodiment, mixed populations of cancerous cells are grown under nonadherent cell culture conditions, wherein NSCs form spherical clusters of cells (“spheres”) from which OCT4 positive NSCs can be enriched. In another embodiment, cells derived from free floating spheres express higher levels of OCT4 and Nanog mRNA than equivalent, adherent cell cultures as shown in FIG. 3. In another embodiment, the cells comprising the spheres are free floating. In another embodiment, in-vitro enrichment of NSCs from breast tumor specimens is carried out using a nonadherent mammasphere cell culture system. In another embodiment, in-vitro enrichment of NSCs from bone sarcoma tumor cells is carried out using a nonadherent sarcosphere cell culture system. In another embodiment, in-vitro enrichment of NSCs from brain tumor cells is carried out using a nonadherent neurosphere cell culture system. In another embodiment, in-vitro enrichment of NSCs from brain tumor cells is carried out using free floating spheres.

In another embodiment, the NSC-enriched subpopulation of cancerous cells is at least 60% positive for OCT4 expression. In another embodiment, the NSC-enriched subpopulation of cancerous cells is at least 70% positive for OCT4 expression. In another embodiment, the NSC-enriched subpopulation of cancerous cells is at least 80% positive for OCT4 expression. In another embodiment, the NSC-enriched subpopulation of cancerous cells is at least 80% positive for Nanog expression. In another embodiment, the NSC-enriched subpopulation of cancerous cells is at least 80% positive for STAT3 expression. In another embodiment, the NSC-enriched subpopulation of cancerous cells is at least 80% positive for the expression of OCT4, STAT 3, Nanog or combinations thereof. In another embodiment, the NSC-enriched subpopulation of cancerous cells is at least 90% positive for OCT4 expression. In another embodiment, the NSC-enriched subpopulation of cancerous cells is at least 90% positive for Nanog expression. In another embodiment, the NSC-enriched subpopulation of cancerous cells is at least 90% positive for STAT3 expression. In another embodiment, the NSC-enriched subpopulation of cancerous cells is at least 90% positive for the expression of OCT4, STAT 3, Nanog or combinations thereof. In another embodiment, the NSC-enriched subpopulation of cancerous cells is at least 95% positive for OCT4 expression. In another embodiment, the NSC-enriched subpopulation of cancerous cells is at least 95% positive for Nanog expression. In another embodiment, the NSC-enriched subpopulation of cancerous cells is at least 95% positive for STAT3 expression. In another embodiment, the NSC-enriched subpopulation of cancerous cells is at least 95% positive for the expression of OCT4, STAT 3, Nanog or combinations thereof.

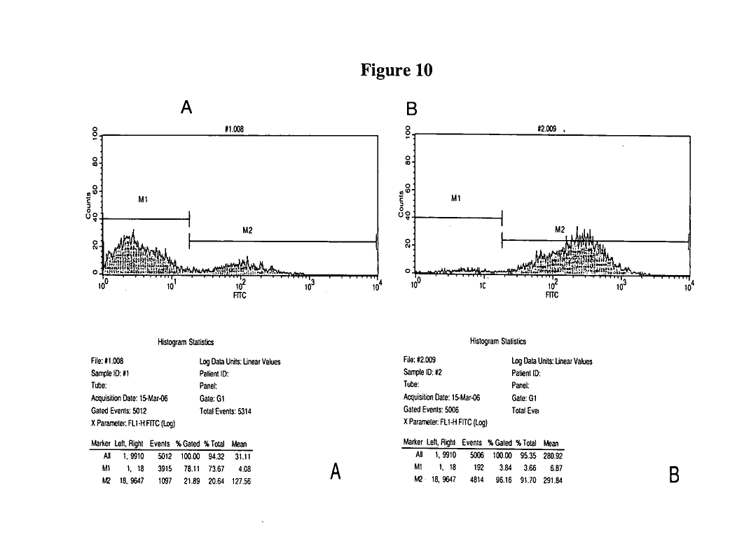

In another embodiment, the invention provides that the level of NSC-enriched subpopulation of cancerous cells is determined by FACS analysis (FIG. 10), in-situ hybridization, immunohistochemistry or a combination thereof, as described in the material and methods section.

In another embodiment, the NSC-enriched population is characterized by OCT4^{h} expression. In another embodiment, OCT4^{hi} expression is at least twice as high as β-actin expression. In another embodiment, OCT4^{hi} expression is at least four times as high as β-actin expression. In another embodiment, the NSC-enriched population is further characterized by high expression of Nanog, STAT3, or combinations thereof.

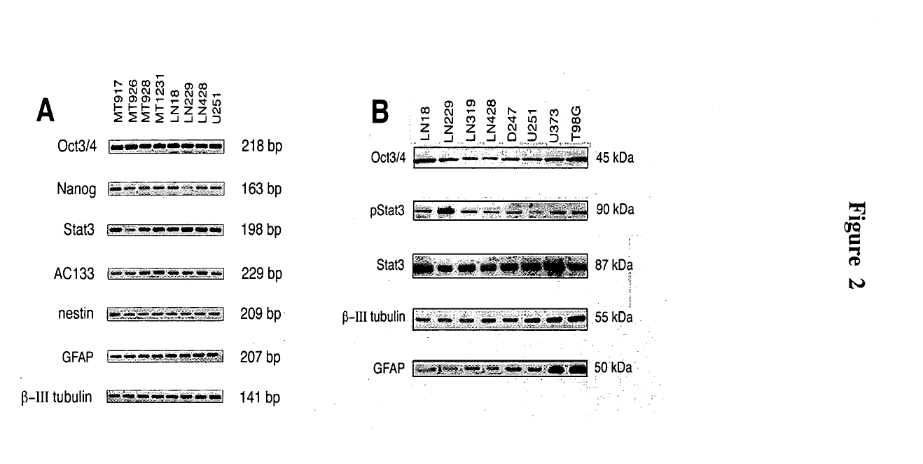

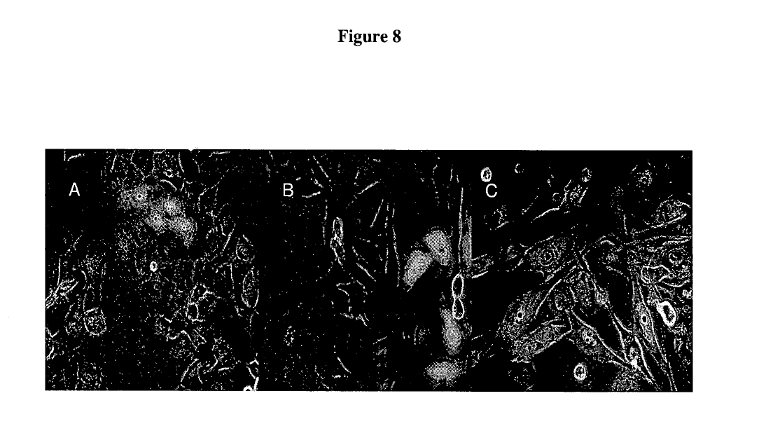

In another embodiment, the expression level of OCT4, Nanog or STAT3 is determined by the mRNA transcription level. In another embodiment, the transcription levels are determined by quantitative or semi-quantitative PCR or RT-PCR methods as shown in FIG. 2A and described in the materials and methods section. In another embodiment, the expression level of OCT4, Nanog or STAT3 is determined by the protein expression level. In another embodiment, the protein expression level is determined by western blot analysis as shown in FIG. 2B and described in the materials and methods section. In another embodiment, protein expression level is determined indirectly by using a reporter gene. In another embodiment, the reporter gene comprises an EGFP construct. In another embodiment, the OCT4 expression level in an OCT4-EGFP transfected glioblastoma cell culture (FIGS. 7 and 8) is determined as described in the materials and methods section.

In another embodiment, the NSC subpopulation is enriched from “soft” or “hard” tumors. In another embodiment, “hard” tumors include all tumors except leukemia, lymphomas, melanomas, and multiple myeloma, which, in another embodiment, are classified as “soft.” In another embodiment, the NSC subpopulation is enriched from isolated metastatic cells. In another embodiment, the NSC subpopulation is enriched from a tissue culture comprising cells derived from a tumor-derived cell line.

In another embodiment, the subject invention comprises a composition comprising a population of NSCs enriched for expression of OCT4. In another embodiment, the invention comprises a population of NSCs enriched for expression of OCT4^{hi}. In another embodiment, the composition further comprises an appropriate environment, such as those described herein, wherein, a NSC can be induced to proliferate and generate NSC progeny. In another embodiment, the term environment in which NSC progeny are placed, refers to the combination of external or extrinsic physical and/or chemical conditions that affect and influence the growth and development of NSCs. In another embodiment, the environment can be ex-vivo or in-vivo. In another embodiment, the circulatory system (blood and lymphatic) can serve as an in-vivo environment that induces NSCs to generate progeny. In another embodiment, the environment is ex-vivo and comprises NSCs placed in cell culture medium in an incubator.

In another embodiment, the environment further comprises cell culture medium comprising DMEM/F12. In another embodiment, the cell culture medium further comprises methylcellulose in a final concentration of less than 3%, more preferably, less than 1.5%. In another embodiment, the medium is supplemented with 8-20% fetal bovine serum (FBS), 30-70% media derived from cultures of primary human foreskin fibroblasts, or a combination thereof. In another embodiment, the medium further comprises screening agents which bind OCT4. In another embodiment, the medium further comprises screening agents which interact with an OCT4 responsive element.

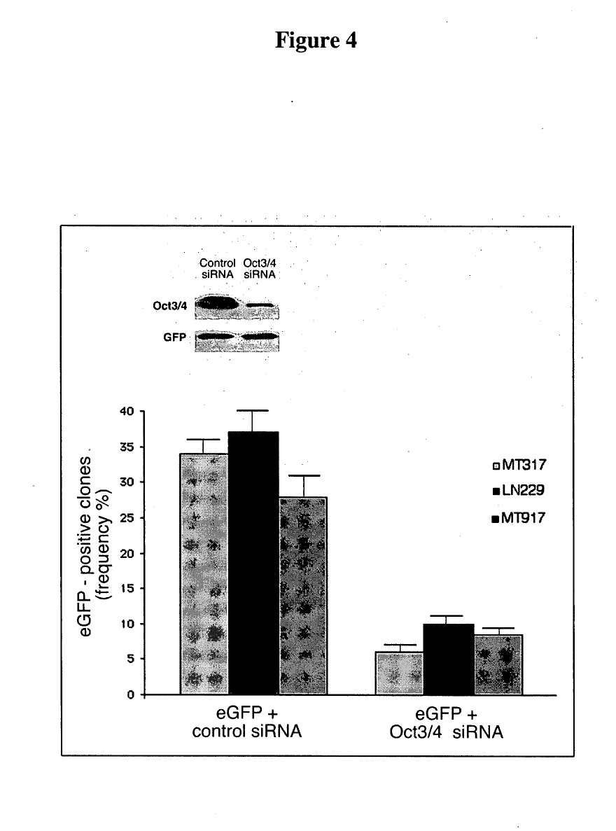

In another embodiment, the medium is further supplemented with 5-50 nM of progesterone, 5-500 μM putrescine, 2-100 ng/ml recombinant EGF, 20-40 nM sodium selenit, 10-40 μg/ml transferring, 5-50 μg/ml insulin. 2-100 ng/ml recombinant FGF2 or a combination thereof. In another embodiment, the medium is supplemented with 8-20% fetal bovine serum (FBS), 30-70% media derived from cultures of primary human foreskin fibroblasts, or a combination thereof. In another embodiment, the medium comprises nucleic acids. In another embodiment, the medium comprises a plasmid DNA. In another embodiment, the plasmid DNA comprises an OCT4 responsive promoter. In another embodiment, the OCT4 responsive promoter is linked to a reporter gene (FIG. 8). In another embodiment, the OCT4 responsive promoter is linker to an antibiotic resistance gene. In another embodiment, the medium comprises siRNA. In another embodiment, the siRNA antisense encodes for anti-OCT4, anti-Nanog, anti-STAT3 or combinations thereof. In another embodiment, the anti-OCT4 siRNA inhibits clone formation (FIG. 4B) by inhibiting de-novo production of OCT4 protein (FIG. 4A).

In another embodiment, cells are plated in ultra low attachment plates. In another embodiment, the cells are kept in an incubator maintaining a temperature at 36-42° C. In another embodiment, the incubator further maintains 4-8% CO₂. In another embodiment, the incubator maintains 90-100% humidity. In another embodiment, cells are plated in a final density of 1×10²-10⁶ cells/cm².

In another embodiment, NSCs of the present invention are derived from a cell line. In another embodiment, NSCs of the present invention are derived from a primary cell culture. In another embodiment, the primary cell culture comprising NSCs is derived from a tumor or cell metastasis. In another embodiment, the invention comprises tumors and cell metastasis which comprise NSCs. In another embodiment, tumors and cell metastasis are derived from but not limited to: adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, ewings family of tumors (pnet), extracranial germ cell tumor, eye cancer, intraocular melanoma, gallbladder cancer, gastric cancer, germ cell tumor, extragonadal gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, small cell, lymphoma, AIDS-related, lymphoma, central nervous system (primary), lymphoma, cutaneous T-cell, lymphoma, hodgkin's disease, non-hodgkin's disease, malignant mesothelioma, melanoma, merkel cell carcinoma, metasatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, sezary syndrome, skin cancer, cutaneous T-cell lymphoma, skin cancer, kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, or wilms' tumor.

In another embodiment, the invention provides a method of identifying NSCs, comprising the steps of contacting neoplastic cells with an agent which specifically interacts with OCT4 through its employment to a cell culture comprising primary cell culture or a cell line culture. In another embodiment, NSCs subpopulation is identified in “soft or hard” tumor. In another embodiment, “Hard” tumors include all tumors except leukemia, lymphomas, melanomas, and multiple myeloma, which are classified as “soft.” In another embodiment, NSCs are identified among metastatic cells.

In another embodiment, the invention provides a method of identifying NSCs, comprising the steps of contacting neoplastic cells with an agent which specifically interacts with OCT4 and identifying the cells with which the agent specifically interacted, as described herein. In another embodiment, the agent identifying OCT4 interacts with the cell membrane. In another embodiment, the agent interacts with the POU5F1 gene encoding OCT4 or a fragment thereof. In another embodiment, the agent interacts with the mRNA encoding OCT4 or a fragment thereof. In another embodiment, the agent interacts with the OCT4 protein or a fragment thereof. In another embodiment, the agent interacts with a specific post translational form of OCT4 such as, but not limited to, the phosphorylated OCT4 protein.

In another embodiment, the invention provides a method of identifying NSCs using a DNA probe that specifically interacts with OCT4 mRNA in a DNA-RNA heteroduplex. In another embodiment, the method of identifying NSCs utilizes an RNA probe that specifically interacts with OCT4 mRNA in an RNA-RNA homoduplex. In another embodiment, the method of identifying NSCs utilizes a peptide nucleic acid (PNA) probe that specifically interacts with OCT4 mRNA in a PNA-RNA heteroduplex. In another embodiment, the nucleic acid probe or PNA further comprises a label which can be readily identified. In another embodiment, the methods utilize a specific probe comprising a nucleic acid that enables selective identification of OCT4 expressing cells.

In another embodiment, the invention provides a method for identification of NSCs comprising a ligand that specifically interacts with OCT4 protein or a fragment thereof. In another embodiment, the invention provides a method for identification of NSCs comprising a ligand that specifically interacts with OCT4 protein or a fragment thereof. In another embodiment, a monoclonal or poyclonal anti-OCT4 antibody is utilized to detect OCT4.

In another embodiment, the invention provides a method of detecting OCT4 expressing cells. In another embodiment, the detection method is direct, wherein a radioactive label is used, which in another embodiment comprises a radioactive compound such as ³²P or ¹²⁵I. In another embodiment, direct labeling comprises a fluorescent, chemiluminescent, or gold label. In another embodiment, the detection method is indirect comprising a nucleic acid probe similar to immunohistochemical probes as known to one skilled in the art. In another embodiment, probes may be labeled with hapten or biotin used to bring an enzyme which creates the detectable event (e.g., chemiluminescent, colorimetric or fluorescent) to the site of hybridization. In another embodiment, wherein amplification of the detection signal is required, a secondary labeled antibody specifically identifying the primary antibody is utilized. In another embodiment, the methods utilizing a specific probe comprising an antibody enable selective identification of OCT4 expressing cells.

In another embodiment, a heterogeneous cell population for OCT4 expression is transfected with a plasmid comprising an OCT4 responsive promoter controlling the expression of an identifiable, reporting gene product. In another embodiment, the identifiable gene product comprises green fluorescent proteins such as but not limited to: GFP, Emerald, Azami Green, or ZsGreen1; blue fluorescent proteins such as but not limited to: EBFP or Sapphire; cyan fluorescent proteins such as but not limited to: Cerulean, ECFP, AmCyan1 or Midoriishi-Cyan; yellow fluorescent proteins such as but not limited to: ZsYellow1, PhiYFP, Citrine, or Venus; orange fluorescent proteins such as but not limited to: Kusabira-Orange or mOrange; red fluorescent proteins such as but not limited to: DsRed, HcRed, mPlum, mRaspberry, mTomato, mStrawberry or green-to-red fluorescent Dendra. In another embodiment, the identifiable gene product serves as a distinguishable marker between cells expressing OCT4 and cells not expressing OCT4 (FIG. 8).

In another embodiment, the invention provides a method of identifying NSCs expressing OCT4, which comprises visualizing the probed NSCs. In another embodiment, visualization of NSCs expressing OCT4 is carried out by exposing the labeled specimen to a film. In another embodiment, visualization of NSCs expressing OCT4 can be performed with a fluorescent microscope. In another embodiment, visualization of NSCs expressing OCT4 can be performed with a confocal microscope. In another embodiment, visualization of NSCs expressing OCT4 can be performed with an electron microscope. In another embodiment, a light microscope is used for visualization of NSCs expressing OCT4, while in another embodiment, the signal is detectable using the naked eye. In another embodiment, the results of the above mentioned visualization methods can be further recorded and/or visualized on a CCD camera.

In another embodiment, the invention provides a method of isolating neoplastic stem cells, comprising the steps of contacting neoplastic cells with an agent which specifically interacts with OCT4. In another embodiment, a cell culture comprising primary cell culture or a cell line culture is employed.

In another embodiment, the invention provides cell separation methods which include cell isolation methods. In another embodiment, tissue dissociation techniques are utilized prior to cell separation methods. In another embodiment, enzymes such as liberase, trypsin, elastase, dispase, collagenase or combinations thereof are employed for effective tissue dissociation. In another embodiment, further trituration with a pipette tip to break apart the cell aggregates is needed.

In another embodiment, the invention provides a method of isolating neoplastic stem cells, comprising the steps of contacting neoplastic cells with an agent which specifically interacts with OCT4 and isolating the cells with which the agent specifically interacts, as described. In another embodiment, the methods described previously for identification of neoplastic stem cells, particularly the steps of contacting neoplastic cells with an agent which specifically interacts with OCT4 protein or mRNA, are also used for isolation of NSCs.

In another embodiment, the invention provides a heterogeneous cell population transfected with a plasmid comprising an Oct4 responsive promoter controlling the expression of an identifiable and/or selectable gene product (FIG. 8). In another embodiment, the methods described previously for identification of neoplastic stem cells comprising the use of various identifiable fluorescent protein sequences are also employed for cell separation methods. In another embodiment, the identifiable gene product is used selectively to isolate OCT4 expressing cells resulting in a uniform OCT4 expressing NSCs.

In another embodiment, NSCs expressing OCT4 are separated in chromatography columns in which antibodies specific to OCT4 that are attached to the column bind OCT4 expressing NSCs and thereby separate them. In another embodiment, an agent that is covalently bound to magnetic particles and that specifically interacts with OCT4 is employed to retain OCT4 expressing NSCs in a magnetic field. In another embodiment, sorting of OCT4 expressing NSCs labeled with antibodies comprising a fluorescent label, through a FACS is used to separate NSCs from a heterogeneous population of cells as shown in FIG. 10. In another embodiment, the separation methods as described herein results in an isolated population of OCT4 expressing cells.

In another embodiment, the invention provides methods of enriching NSCs expressing OCT4. In another embodiment, a primary cell culture is enriched for OCT4 expressing cells. In another embodiment, the primary cell culture for which methods for enriching OCT4 expressing NSCs is employed is derived from a soft tumor, a hard tumor, or a metastatic cell population. In another embodiment, the OCT4 expressing NSC subpopulation is enriched from a tissue culture comprising cells derived from a cell line.

In another embodiment, the invention provides methods of enriching OCT4 expressing NSCs which comprise transfection of a heterogeneous cell population with a plasmid comprising an Oct4 responsive promoter controlling the expression of a selectable gene product (FIG. 8). In another embodiment, the selectable gene encodes an antibiotic resistance protein. In another embodiment, the cell enrichment methods further comprise the selecting agent. In another embodiment the selecting agent is an antibiotic which selectively eradicates non-OCT4 expressing cells resulting in an enriched OCT4 expressing NSC cell population.

In another embodiment, the invention provides a method of inducing cancer comprising introducing a neoplastic stem cell population enriched for expression of OCT4 to a mammal. In another embodiment, the method of inducing cancer comprises promoting cell growth that leads to cancer. In another embodiment, the method of inducing cancer comprises providing metastatic cells that induce cancer.

In another embodiment, NSCs of the invention isolated from mamaspheres, sarcospheres or neurospheres are used as cancer inducers. In another embodiment, an animal is inoculated with NSCs. In another embodiment, NSCs are injected intravenously. In another embodiment, NSCs are injected into the bone. In another embodiment, NSCs are injected into an animal intradermally, intramuscularly or intraperitoneally. In another embodiment, NSCs are injected directly to the mammary gland of a model animal. In another embodiment, inoculation comprises injection of NSCs into the fat pads of a model animal.

In another embodiment, the invention provides methods of inducing cancer. In another embodiment, the methods of inducing cancer as described herein are performed in immunodeficient rodents. In another embodiment, the immunodeficient rodent is a nude mouse or rat. In another embodiment, the immunodeficient rodent is a SCID mouse. In another embodiment, the immunodeficient rodent is an NIH-III mouse.

In another embodiment, the invention provides a method of inducing tumors or metastases comprising introducing a neoplastic stem cell population enriched for expression of OCT4 to a mammal. In another embodiment, orthotopical or ectopical tumors are being induced (FIG. 11). In another embodiment, metastases take place through the lymphatic system, through the bloodstream, by spreading through body spaces, or through implantation.

In another embodiment, the invention provides a method of analyzing cancer progression and/or pathogenesis in-vivo comprising transplanting OCT4^{hi} neoplastic stem cells into an animal; and analyzing cancer progression and/or pathogenesis in an animal. In another embodiment, cancer comprises carcinoma, sarcoma, lymphoma, leukemia, or myeloma.

In another embodiment, NSCs of the invention are labeled by transfecting OCT4^{hi} neoplastic stem cells with a fluorescent protein. In another embodiment, the identifiable gene product comprises various fluorescent proteins as described hereinabove. In another embodiment, the identifiable gene product comprises a luminescent protein. In another embodiment, the luminescent protein is luciferase.

In another embodiment, isotopes are used for tracking the transplanted OCT4^{hi} neoplastic stem cells in the animal model. In another embodiment, the isotopes comprise ³²P, ¹²⁵I, ¹²⁴I, ¹²³I, ¹⁴C, ¹⁰⁹Cd, ⁵¹Cr, ⁶⁷Cu, ¹⁷⁹Ta, ¹¹¹In, ¹⁸F, or combinations thereof. In another embodiment a magnetic label is used for cell detection.

In another embodiment, the transplanted labeled cells of the invention were tracked with a single-photon emission-computed tomographic (SPECT) scanner, a positron emission tomography (PET) scanner, or single photon emission commuted tomography. In another embodiment, wherein cells are labeled magnetically, MRI is used for detection. In another embodiment, a back-illuminated, cooled, charge-coupled device (CCD) camera is used for luminescent detection. In another embodiment, LED flashlights with excitation filter and an emission filter are used for detection of fluorescently labeled cells. In another embodiment, light box with fiber-optic lighting at about 490 nm and filters, placed on top of the light box, are used to image large tumors. In another embodiment, small tumors and metastases are visualized using a fluorescence dissecting microscope that incorporates a light source and filters for excitation at about 490 nm. In another embodiment, color CCD cameras as well as dual-photon lasers are used for ultra-high-resolution in-vivo imaging of fluorescent protein expression.

In another embodiment, the invention provides a method of analyzing cancer progression and/or pathogenesis in-vivo including determining cell metastasis. In another embodiment, analysis of cell metastasis comprises determination of progressive growth of cells at a site that is discontinuous from the primary tumor. In another embodiment, the site of cell metastasis analysis comprises the route of neoplastic spread. In some embodiment, cells can disperse via blood vasculature, lymphatics, within body cavities or combinations thereof. In another embodiment, cell metastasis analysis is performed in view of cell migration, dissemination, extravasation, proliferation or combinations thereof.

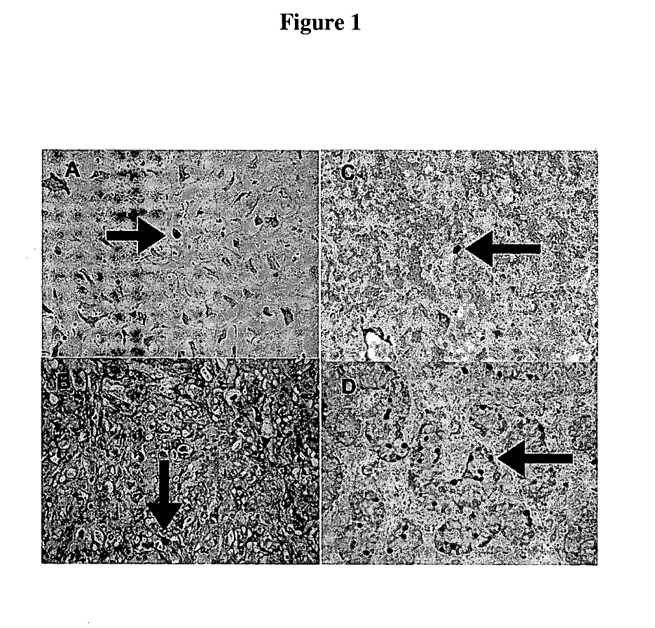

In another embodiment, the invention provides a method of analyzing cancer progression and/or pathogenesis in-vivo. In another embodiment, analysis of cancer progression and/or pathogenesis in-vivo comprises determining the extent of tumor progression. In another embodiment, analysis comprises the identification of the tumor (FIG. 11). In another embodiment, analysis of tumor progression is performed on the original tumor or “primary tumor”. In another embodiment, analysis is performed over time depending on the type of cancer as known to one skilled in the art (FIG. 11). In another embodiment, further analysis of secondary tumors originating from metastasizing cells of the primary tumor is analyzed in-vivo. In another embodiment, the size and shape of secondary tumors are analyzed. In some embodiment, further ex-vivo analysis is performed. In another embodiment, the frequency of OCT4 expressing cells in chondrosarcoma or oteosarcoma tumors is assessed as shown in FIG. 1.

In another embodiment, the terms assessed, screened, evaluated and analyzed are used interchangeably.

In another embodiment, pathological samples of metastasis or tumors are evaluated at specific points in time, as known to one skilled in the art. In another embodiment, quantitative or qualitative methods assessing tumor suppressor genes, oncogenes, apoptotic genes, signal transduction genes, receptors, transcription factors, ligands or combinations thereof comprising: PCR, western-blot, northern blot, southern blot, immunohistochemical or in situ hybridization analysis are further employed.

In another embodiment, tumor or metastatic cells are isolated from pathological samples for further analysis. In another embodiment, tumor or metastatic cells are isolated from pathological samples and grown in culture. In another embodiment, the cell proliferation potential of the primary tumor cell culture is assessed. In another embodiment, OCT4 positive cells are isolated and/or enriched from the pathological sample comprising tumor or metastatic cells according to the methods described hereinabove. In another embodiment, the OCT4 positive cells isolated from a tumor are further analyzed. In some embodiment, various agents are further employed to the tumor or metastasis primary cell culture. In another embodiment, the agent is a carcinogen. In another embodiment. The agent is a pro-apoptotic agent or a differentiating agent.

In another embodiment, the invention provides a method of assessing the effect of a carcinogen on a primary cell culture. In another embodiment, the carcinogen comprises, but is not limited to, carcinogenic substances in categories 1 through 3 of the International Agency for Research on Cancer (IARC).

In another embodiment, the invention provides a method of assessing the effect of a therapeutic agent on a primary cell culture derived from a tumor or a metastasis. In another embodiment, therapeutic agents are screened ex-vivo, on a tumor or metastasis-derived primary cell culture. In another embodiment, the therapeutic agents comprise interferons, interleukins, colony-stimulating, alkylating agents, nitrosoureas, antimetabolites, antitumor antibiotics, plant (vinca) alkaloids, steroid hormones or combinations thereof. In another embodiment, the therapeutic agent is a chemotherapy agent. In another embodiment, the chemotherapy agent is non-specific and hence may kill a cancerous cell during any phase of the cell-cycle. In another embodiment, the chemotherapy agent is specific and is thus able to kill a cancerous cell during a specific phase of the cell-cycle.

In another embodiment, the present invention provides that heterogeneous cancer cell populations derived from clinical tumor specimens (whether primary or metastatic) or from permanent tumor cell lines can be manipulated to allow for the isolation and propagation of their respective cancer stem cell populations. In another embodiment, the present invention provides methods for the identification, sorting and stable maintenance in culture subsets of NSCs based on their ability to maintain the expression of fluorescent (or luminescent) proteins driven by the promoter of the Oct3/4 transcription factor. In another embodiment, the present invention provides that Oct3/4 transcription factor in concert with SOX-2, Nanog and STAT3, are the regulators of normal stem cell phenotype in the context of embryonic development including the process of self-renewal.

In another embodiment, the present invention provides that in the context of cancer, NSCs do not have the appropriate proliferation controls allowing the process of self-renewal to go unchecked resulting in dysplastic tissue mass at site of proliferation. In another embodiment, the methods of the present invention provide the use of biomarkers that are regulated in parallel to the molecular machinery mentioned above. In another embodiment, these regulated biomarkers monitor the “stemness” of a given cancer cell.

In another embodiment, the present invention allows for the monitoring of the relative viability and “stemness” of the NSC population.

In another embodiment, the present invention provides that NSCs are responsible for metastasis to systemic organs. In another embodiment, the present invention provides that NSCs are required for metastasis to systemic organs. In another embodiment, the present invention provides that NSCs are responsible for recurrent cancer growth in the primary location after attempts at treatment (i.e., surgery, radiation, and chemotherapy). In another embodiment, the methods of the present invention provide a platform which allows for the identification of drugs that target those NSCs with metastatic potential.

In another embodiment, the method of the present invention is carried out using cells cultured in miniaturized format. In another embodiment, cells cultured in miniaturized format of the present invention comprise multi-well plates. In another embodiment, a multi-well plate of the present invention comprises 96 wells. In another embodiment, a multi-well plate of the present invention comprises 384 wells (example 2). In another embodiment, a multi-well plate of the present invention comprises 1536 wells. In another embodiment, a multi-well plate of the present invention comprises from 2-5000 wells. In another embodiment, a multi-well plate of the present invention comprises from 20-3000 wells. In another embodiment, a multi-well plate of the present invention comprises from 96-2000 wells.

In another embodiment, the invention provides a method of evaluating the effect of photodynamic therapy (PDT) on tumor derived primary cell culture. In another embodiment, the effect of radiation therapy or radiofrequency ablation alone or in combination with any other form of a therapeutic agent on tumor primary cell culture is further assessed. In another embodiment, the effect of chemoembolization on tumor derived primary cell culture is analyzed. In another embodiment, the effect of local hyperthermia on tumor derived primary cell culture is analyzed. In some embodiment, the in-vivo effect of various agents and conditions is desired.

In another embodiment, the invention provides a method wherein an agent of interest is further administered in-vivo to an animal that has been transplanted with OCT4 expressing NSCs. In another embodiment, the OCT4 expressing NSCs express OCT4^{hi}. In another embodiment, administration of an agent is according to procedures known to one skilled in the art. In another embodiment, single or multiple administrations of an agent or agents are required, as known to one skilled in the art. In another embodiment, the agent or agents are administered over a period of days to weeks or over a period of months to years, depending on cancer progression and/or regression, as known to one skilled in the art. In another embodiment, the agent is a carcinogen which in another embodiment is a carcinogenic substance in categories 1 through 3 of the International Agency for Research on Cancer (IARC). In another embodiment, the agent is a therapeutic agent.

In another embodiment, the invention provides a means of exploring the effects of a therapeutic agent on cancer progression (FIG. 12). In another embodiment, the effects of a therapeutic agent on cell metastasis potential are evaluated. In another embodiment, the effects of a therapeutic agent on a soft tumor are evaluated. In another embodiment, the effects of a therapeutic agent on a hard tumor are evaluated. In another embodiment, the effect of a therapeutic agent on primary and/or secondary tumor growth is evaluated.

In another embodiment, the therapeutic agent or agents administered in-vivo to an animal transplanted with OCT4 or OCT4^{hi} neoplastic stem cells (FIG. 12) comprise: interferons, interleukins, colony-stimulating, alkylating agents, nitrosoureas, antimetabolites, antitumor antibiotics, plant (vinca) alkaloids, steroid hormones or combinations thereof. In another embodiment, the therapeutic agent is a chemotherapy agent. In another embodiment, the chemotherapy agent is non-specific and therefore has the potential to kill a cancerous cell during any phase of the cell-cycle. In another embodiment, the chemotherapy agent is specific and thus is able to kill cancerous cells during a specific cell cycle phase.

In another embodiment, the in-vivo effect of PDT on an animal transplanted with OCT4^{hi} neoplastic stem cells is evaluated. In another embodiment, the in-vivo effects of radiation therapy or radiofrequency ablation alone or in combination with any other form of a therapeutic agent in-vivo are further assessed. In another embodiment, the in-vivo effects of chemoembolization or local hyperthermia, on cancer progression and/or regression are evaluated.

In another embodiment, the in-vivo effect of biological therapy on an animal transplanted with OCT4^{hi} neoplastic stem cells derived from tumor or primary cell culture is analyzed. In another embodiment, biological therapies comprise immunotherapy. In another embodiment, immunotherapy comprises the use of a vaccine comprising immunogenic fragments derived from Nanog, STAT3, OCT4, or combinations thereof, as described hereinabove. In another embodiment, the effect of nonspecific immunomodulating agent or agents is assessed. In another embodiment, the nonspecific immunomodulating agent is bacillus Calmette-Guerin (BCG) or levamisole.

In another embodiment, OCT4 modifiers are screened in-vivo for cancer progression or regression in an animal transplanted with OCT4^{hi} neoplastic stem cells (FIG. 12). In another embodiment, OCT4 monoclonal antibodies are screened in-vivo. In another embodiment, intrabodies specific to an OCT4 protein are screened in-vivo. In another embodiment, PNAs, aptamers, or antisense siRNA are further evaluated in-vivo as shown in FIG. 4.

In another embodiment, the invention provides a method of preventing, treating, abrogating, or inhibiting cancer, tumor growth, cell metastasis or combinations thereof comprising the step of contacting neoplastic cells with an agent that inhibits OCT4 expression or function. In another embodiment, OCT4 is inhibited transiently. In other embodiments, OCT4 is inhibited constitutively.

In another embodiment, the invention provides a method of inhibiting OCT4 comprising targeting OCT4 expression at the DNA level and thus inhibiting or abrogating OCT4 transcription. In another embodiment, inhibition of OCT4 at the DNA level is accomplished via the formation of DNA triple-stranded structures. In another embodiment, the triple helix inhibition complex, is designed as an OCT4 gene-specific oligonucleotide and thus inhibits OCT4 transcription.

In another embodiment, the invention provides a method of inhibiting OCT4 comprising targeting OCT4 expression at the RNA level and thus inhibiting OCT4 expression. In another embodiment, RNA is mRNA. In another embodiment, antisense based therapeutics are used to inhibit OCT4. In another embodiment, synthetic oligonucleotides are designed to be complementary in sequence to a specific OCT4 mRNA sequence and thus inhibit OCT4 expression.

In another embodiment, the invention provides peptide nucleic acids (PNAs) that are artificially constructed to hybridize to an OCT4 mRNA sequence and thus inhibit OCT4 expression. In another embodiment, the binding agent is a specifically engineered ribozyme, which cleaves OCT4 mRNA transcripts and subsequently inhibits OCT4 expression.

In another embodiment, the method of inhibiting OCT4 function comprises targeting OCT4 protein. In another embodiment, inhibition of OCT4 function is achieved through the specific binding of an antibody to an OCT4 protein and thus inhibiting or abrogating OCT4 protein binding to an OCT4 responsive DNA element. In another embodiment, intrabody or antibodies raised subsequent to OCT4 immunotherapy, inhibit or abrogate OCT4 function.

In another embodiment, the invention provides an intrabody specific to OCT4 protein. In another embodiment, intrabodies comprise a single chain of a coupled variable domain of the heavy chain to the variable domain of the light chain through a peptide linker and are used to interfere with the binding of the OCT4 protein to an OCT4 DNA responsive element. In another embodiment, intrabodies are directed to the cell nucleus where they inhibit or abrogate binding of an OCT4 protein to an OCT4 DNA responsive element. In another embodiment, the intrabodies target the OCT4 protein DNA binding domain on the OCT4 protein and hence, inhibit or abrogate OCT4 protein binding to an OCT4 DNA responsive element.

In another embodiment, the invention provides a vaccine comprising an OCT4 peptide. In another embodiment, the OCT4 peptide elevates OCT4 specific antibodies. In another embodiment, the peptide consists of the full length OCT4 gene. In another embodiment, the peptide is a mutated form of OCT4. In another embodiment, a 4-18 amino acid long OCT4 peptide is used. In another embodiment, the vaccine comprises OCT4 peptides of uniform length and sequence. In another embodiment, the vaccine comprises a mixture of OCT4 peptides that differ in both length and sequence.

In another embodiment, oligonucleotide aptamers are used to bind specific OCT4 protein sequence and thus inhibit or abrogate OCT4 protein binding to an OCT4 DNA responsive element.

In another embodiment, the invention provides a mutated OCT4 protein. In another embodiment, the mutated OCT4 protein is used to block the transcription of downstream OCT4 responsive genes. In another embodiment, the mutated OCT4 protein used to inhibit or abrogate OCT4 responsive gene expression and the wild type OCT4 protein have similar affinities to the OCT4 DNA responsive element. In another embodiment, the mutated OCT4 protein used to inhibit or abrogate OCT4 responsive genes expression have a higher affinity to the OCT4 DNA responsive element compared to the wild type OCT4 protein.

### Materials and Methods

Immunohistochemical Identification of OCT4, STAT3 or Nanog Positive Cells in Tissue

Immunohistochemical staining for OCT4, STAT3 or Nanog histological analyses of tumor biopsies from human and from mice were preformed as follows: Formalin fixed paraffin embedded tissue sections (5 μm) were sequentially deparaffinized, rehydrated and blocked for endogenous peroxidase activity following a 95° C. degree, 25 minute antigen retrieval in Trilogy unmasking solution (Cell Marque, Hot Springs Ark.). Slides were biotin blocked, serum blocked and immunostained using a goat ABC Elite Kit (Vector Labs, Burlingame, Calif.) Antibodies to OCT 3/4, STAT3 and Nanog (R&D Systems, Minneapolis, Minn.) were applied at 1:50 dilution for one hour at room temperature. Positive staining was detected with DAB (3,3′-Diaminobenzidene) and light green SF yellowish or hematoxylin (Sigma, St. Louis, Mo.) was used as counterstain. Alternatively, primary breast cancer foci and the brain and lung were harvested for sectioning by cryostat. Tissues were cut on a cryostat at 16 μm to generate sets that are in the axial plane (breast and lung) and coronal plane (brain). Hematoxylin-eosin (H&E) staining was performed on one set, and immunohistochemistry on a second set. The immunohistochemistry was performed using the following procedures. The frozen breast, lung, and brain sections were (1) incubated in 2% non-fat milk and 0.3% Triton-X in PBS for 1 hour; (2) incubated in OCT4, STAT3 or Nanog antibodies in 3% donkey serum and 0.1% Triton-X overnight at room temperature; (3) washed with PBS for 3 times; (4) incubated with secondary antibody for 4 hours in dark at room temperature; (5) washed with PBS for 3 times; and (6) dehydrated through graded ethanol, cleared with xylene, and coverslipped with DPX mounting medium (44581, Fluka Biochemika). Immunoreactivity was visualized with a Bio-Rad confocal microscope and images collected on a computer for later analysis.

Immunocytochemical Identification of OCT4, STAT3 or Nanog Positive Cell Culture

Immunohistochemical staining for identification of OCT4, STAT3 or Nanog positive cells in primary culture of tumor biopsies from human and mice was carried out by mincing the specimens into small particles in DMEM/F12 medium digested with 300 U/ml Collagenase Type II (Gibco BRL Invitrogen Corporation, Grand Island, N.Y., USA) for 3-6 hours and passed through a 70 μm Cell Strainer (Becton Dickinson Lab Ware, Franklin Lakes, N.J., USA) to prepare single-cell suspension. Cells were then drained of all medium rinsed with PBS, suspended in culture medium and plated. After cells attachment fixation solution containing fresh 4% formaldehyde solution with 0.1% Triton X-100 was applied. Cells were blocked for endogenous peroxidase activity following biotin blocked, serum blocked and immunostained using a goat ABC Elite Kit (Vector Labs, Burlingame, Calif.) Antibodies to OCT 3/4, STAT3 and Nanog (R&D Systems, Minneapolis, Minn.) were applied at 1:200 for 30 minutes at room temperature. Positive staining was detected with DAB (3,3′-Diaminobenzidene) and hematoxylin (Sigma, St. Louis, Mo.) was used as the counterstain.

The OCT4-EGFP and Nanog-EGFP Constructs

The OCT4-EGFP and Nanog-EGFP constructs were engineered using strategies and techniques previously described (Gerrard et al., 2005). A plasmid containing the EGFP reporter (pEGFPI, BD Biosciences) and the selectable marker G418 under the control of the OCT4 and Nanog promoter was used. The promoter fragment of human OCT4 spans from base −3917 to base +55 of the OCT4 gene (hOCT4pr, from 67539 to 71490 in the human DNA sequence), and contains two appropriate regulatory elements which drove developmentally specific EGFP expression. The promoter fragment of human Nanog spans from base −132 to base +300 (from base 697969 to base 701269 in the human genomic DNA sequence of Chromosome 12).

Expression Vectors Used to Create Biomarked Cells

Oct4hP-eGFP plasmid was constructed using the human Oct-4 responsive promoter (Oct4hP, from 67539 to 71490 in human DNA sequence with accession number AP000509) that was amplified by polymerase chain reaction with primers Oct4hP-F (5′-TT CCC ATG TCA AGT AAG TGG GGT GG-3′) and Oct4hP-R (5′-CGA GAA GGC AAA ATC TGA AGC CAG G-3′) using human genomic DNA (Promega G3041) as a template. The fragment was cloned into a TOPO vector (Invitrogen) and the fidelity of the DNA sequence was confirmed with bi-directional DNA sequencing. Oct4hP was then cloned into the expression vector pEGFPI (Clontech Cat # 6086-1, Genbank Accession # U55761) by insertion into the HindIII and BamH1 sites upstream of eGFP (FIG. 12A).

Modification of the Neurosphere Culture System for Isolation and Analyses of NSCs from Glioblastoma, Bone Sarcomas, and Breast Cancer

The neurosphere culture system proposed by Weiss and Raynolds (1992) was modified by inhibiting the potential of cells for substrate attachment by exploiting pleiotropical growth factors—EGF, FGF2 and insulin in semi-solid methylcellulose (MC). In this experiment NSCs were transfected with EGFP reporter plasmid having the EFGP and G418 genes under the human promoter of OCT4 as shown in FIG. 7.

Lesions and Foci Dissections

The lesions and foci were dissected from their respective tissue locations using a Leica MZ16FA dissecting microscope with a GFP3 filter (for fluorescence capability) and Q-imaging Retiga EXi monochrome digital camera with RGB filter for in-vitro studies and molecular analysis. This tumor material was further studied by using different assays.

RNA Isolation and Target cDNA Amplification

Total RNA was isolated using the RNeasy Mini Kit (and treated with RNAase-Free DNase Set (Qiagen Sciences, Md., USA), by using DNAase treatment. A SuperScript II RNase H⁺ Reverse Transcriptase first-strand synthesis system (InVitrogen Life Technologies, Carlsbad, Calif.) was used to synthesize the cDNA, from 1.5 μg of total RNA by priming with Oligo(dT)₁₂₋₁₈ (Invitrogen Life Technologies, Carlsbad, Calif.). The target cDNA was amplified by using Platinum TaqDNA Polymerase (InVitrogen Life Technologies, Carlsbad, Calif.) and 37 cycles of PCR. Primers for human beta tubulin III amplified 1356-1497 bp product transcribed from the non-translated 3′ UTR-region of the Hbeta4 gene as described in (Kavallaris et al., 1997). The primers for human OCT3/4 (Accession # Z11898), Nanog (NM 024865), Stat3 (NM 139276), Gata-4 (NM 002052), -6 (NM 005257), AFP (NM 001134), Runx 1 (NM 001754), were originally generated by using the Oligo5.1 program. All primers are provided in Table 1.

TABLE 1
Primers used for gene expression analysis by RT PCR

| | | | Product length |
|---|---|---|---|
| Gene | Forward Primer | Reverse Primer | (bp) |
| GATA4 | GCCCAAGAACCTGAATAAATCTAAG | AGACATCGCACTGACTGAGAACGTC | 208 |
| GATA6 | TTCCCCCACAACACAACCTACAG | GTAGAGCCCATCTTGACCCGAATAC | 118 |
| AFP | GGTGTAGCGCTGCAAACGATG | AATTTAAACTCCCAAAGCAGCACGA | 210 |
| STAT3 | GGGTGGAGAAGGACATCAGCGGTAA | GCCGACAATACTTTCCGAATGC | 198 |
| RUNX1 | CTCAGGTTTGTCGGTCGAAGTGGAA | CCGCAGCTGCTCCAGTTCAC | 216 |
| Nanog | GCTGAGATGCCTCACACGGAG | TCTGTTTCTTGACTGGGACCTTGTC | 163 |
| OCT 3A/4 | TGGAGAAGGAGAAGCTGGAGCAAAA | GGCAGATGGTCGTTTGGCTGAATA | 186 |
| NESTIN | CAGCTGGCGCACCTCAAGATG | AGGGAAGTTGGGCTCAGGACTGG | 209 |
| β-III TUBULIN | CTGCTCGCAGCTGGAGTGAG | CATAAATACTGCAGGAGGGC | 141 |
| GFAP | GCTCGATCAACTCACCGCCAACA | GACCTGACAGACGCTGCTGCCC | 207 |

Western Blot Analysis

Cells were dissolved in lysing buffer containing 50 mM Tris-HCl, 150 mM NaCl, 1 mM EDTA, 1% NP40, 0.1% SDS, 1% Na-deoxycholate, 1 mM Na-vanadate, and protease inhibitors: 5 μg/ml pepstatin, 1 mM phenylmethylsulphonylfluoride, 10 μg/ml leupeptin, 1 mM NaF (Sigma Chemical Co., St. Louis, Mo.) for at least 1 hour on ice. After centrifugation (12,000 g for 10 min at 4° C.), the protein concentration of the supernatant was measured by BCA Protein Assay kit (Pierce, Rockford, Ill.) using Benchmark Microplate Reader (Bio Rad Laboratories, Hercules, Calif. USA). Lysates were mixed (1:1) with Laemmli Buffer (Sigma Chemical Co., St. Louis, Mo.). 15 μg of protein was loaded per lane of 8-16% or 10-20% Tris-HCl Ready Gels (Bio Rad Laboratories, Hercules, Calif.) and separated by electrophoresis. The nitrocellulose membranes (Sigma Chemical Co., St. Louis, Mo.) with transferred proteins was blocked as the manufacture recommended and incubated over night with shaking at 4° C. with the corresponding primary antibodies against: STAT3 (R&D Systems), phospho-Tyr705 Stat3 (Cell Signaling Technology, Ill.), beta-III tubulin (BAbCO, Berkeley, Calif.), beta-actin (Sigma Chemical Co., St. Louis, Mo.), alpha fetoprotein (Santa Cruz Biotech, Calif.), OCT-3/4 (Santa Cruz Biotech., CA, Nanog from (R&D systems), diluted in solution containing 5% bovine albumin, (Sigma Chemical Co., St. Louis, Mo.) in Tris-Buffered Saline (TBS) and 0.1% Tween-20 (Bio-Rad Laboratories, Hercules, Calif. USA). After washing in TBS with 0.1% Tween-20, the blots were incubated with secondary peroxidase-conjugated goat antibodies to mouse or rabbit IgG (Cell Signaling Technology, Ill.) or rabbit antibodies to goat IgG (Jackson Immuno Research Laboratories, West Grove, Pa.). Immunoreactive bands were detected by ECL+ Western Blotting Detection Reagents (Amersham Biosciences, UK) for 60 seconds or more and exposed to X-ray films.

Pre and Post Transfection Cell Culture Media

Cells were cultured in DMEM/F12 medium supplemented with 10% (volume/volume) of characterized fetal bovine serum (FBS) (HyClone, Logan, Utah USA) at 37° C., 7.0% CO₂. All cells were transfected by electroporation with a Gibco Cell Porator pulser. The cell suspension was supplemented with 10 μg of plasmid DNA, and transfected according to protocols known to one skilled in the art (FIG. 11C). After electroporation, a whole suspension was plated at a density of 60,000 cells/2 ml/well in DMEM/F12 with 0.8% of MC, supplemented with progesterone (20 nM), putrescine (100 μM), sodium selenite (30 nM), transferrin (25 μg/ml), insulin (20 μg/ml) (Sigma Chemical Co., St. Louis, Mo. USA) and the growth factors EGF (10 ng/ml) and recombinant FGF2 (10 ng/ml).

Selection of Transfected EGFP—Mammasphere Clones

To select transfected EGFP—mammasphere clones, G418 was added after 3 days of culturing (200 mg/ml). In plates with G418, only green EGFP-positive clones were generated and collected for further manipulations. The generated EGFP-positive mammasphere were used for establishing EGFP-subpopulation with stable integration of EGFP. The green mammasphere expressing EGFP under the control of the Oct4 promoter grew as un-attached suspended mammasphere (FIG. 7B). EGFP cells were isolated directly under fluorescent microscope (FIG. 11D).

### EXAMPLE 1

### The Frequency of OCT4 Expressing Cells in Tumors

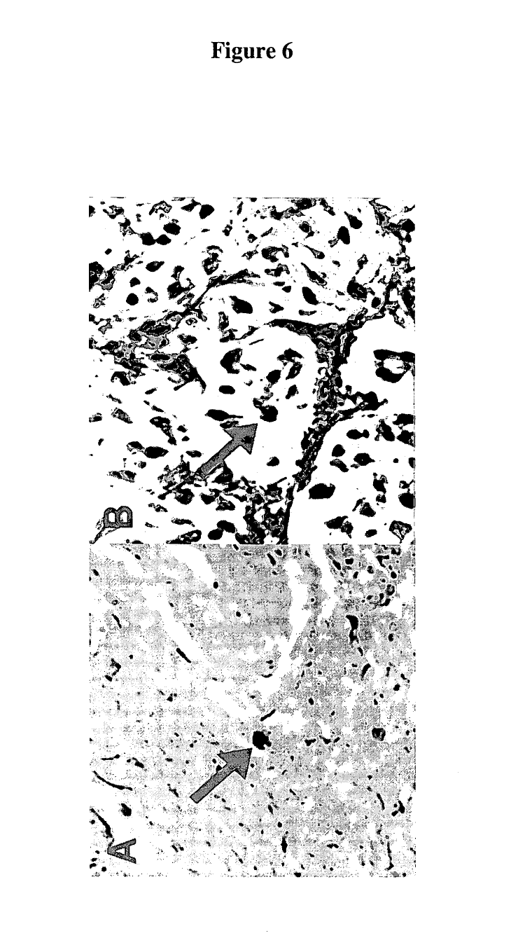

In order to better explore the frequency of OCT4 expressing cells in tumors, immunohistochemical analysis was performed on serial sections derived from: osteosarcoma tumor, glioblastoma tumor, and ductal carcinoma. The results in FIG. 1 indicate that OCT4 positive nuclei are present in osteosarcoma and glioblastoma tumors; furthermore, the results in FIG. 6A-B indicate that OCT4 positive nuclei are also present in ductal carcinoma and breast cancer metastasis to the brain, respectively. Although, OCT4-positive cells were observed in both ductal carcinoma and breast cancer metastasis, more frequent OCT4 positive nuclei were indicated in breast cancer metastasis to the brain (FIG. 6B), compared to primary breast cancer embodied in ductal carcinoma (FIG. 6A). Both tumors and metastasis comprise OCT4-positive cells, thus the methods as described herein provide that these OCT4-positive cells are valid target in cancer therapy.

### EXAMPLE 2

### OCT4, Nanog, and STAT3 are Expressed in Concert in Glioblastomas Clinical Specimens and Cell Lines

In order to determine whether OCT4, Nanog and STAT3 are co-expressed in glioblastomas clinical specimens and cell lines LN18, LN229, LN428 and U251 semi-quantitative RT-PCR analysis followed by western blot analysis was performed. The results as indicated in FIG. 2A show a moderate to high mRNA expression of OCT4, Nanog and STAT3. The protein expression levels were in correlation with the mRNA expression levels as shown in FIG. 2B, wherein, moderate to high protein expression levels of OCT4, Nanog and STAT3 are exhibited.

### EXAMPLE 3

### OCT4 Expression in Tumor Cells Grown Attached or Unattached to a Substrate

In order to test the impact of cell-substrate attachment on OCT4 expression in tumor cells, bone sarcoma and mammary tumor cells were grown attached to a substrate or un-attached as sarcospheres or mammasphere, respectively. The results as shown in FIG. 3 indicate that hi OCT4 and Nanog expression is dependent on cell attachment and thus, tumor cells grown unattached in sarcospheres and mammasphere highly expresses OCT4 and Nanog, in contrast to their suppression in substrate-attached tumor cells wherein the expression of Nanog and OCT4 is relatively low.

### EXAMPLE 4

### OCT4 Role in Clone Generation from Glioma-Derived Tumor Stem Cells Cultured in a Neurosphere System

To test the functional role of OCT4 gene in maintenance of self-renewal in a model culture system, siRNA silencing of POU5F1/OCT4 gene was assessed (FIG. 4). Toward this end, tumor cells derived from three representatives of glioblastoma cell types, including two cell lines and a primary tumor culture isolated from a patient, were co-transfected with EGFP and OCT4 siRNA, and plated in a neurosphere culture system. In a set of parallel control experiments, cells were co-transfected with EGFP and scrambled control siRNA. Clones transfected with EGFP and siRNAs became visible on the 7th day after plating (FIG. 4A). The proportion of clones with detectable EGFP, formed by cells transfected with OCT4-siRNA, fell by more than 60% when compared to controls (FIG. 4B). These findings demonstrated that down regulation of endogenous OCT4 by siRNA in plated tumor-derived cells under growth constraining conditions of a neurosphere culture system results in a significant decrease of clone-formation by tumor-derived EGFP-containing cells.

### EXAMPLE 5

### Tagging MDA MB 231 Breast Cancer Cell Line with EGFP to Asses NSCs Involvement in Orthotopic Tumor Formation

The potential of neoplastic stem cells derived from breast cancer cell line-MDA MB 231 stably expressing EGFP under Oct4 promoter in nude mice was assessed through NSCs involvement in orthotopic tumor formation in nude mice. This assay is based on the presumption that EGFP expression correlates with endogenous OCT4 gene expression. OCT4 positive and OCT4 negative MDA MB 231 breast cancer cells transfected with OCT4-EGFR construct and sorted by FACS were inoculated into fat pad of twelve nude mice which were grouped in four groups each group comprising 3 animal: Group 1 was inoculated with 5,000 OCT4 positive cells/animal, Group 2 was inoculated with 50,000 OCT4 positive cells/animal, Group 3 was inoculated with 500,000 OCT4 positive cells/animal, Group 4 was inoculated with 500,000 OCT4 negative cells/animal. The graph in FIG. 11 shows that animals inoculated with OCT4-positive cells began developing tumors on the 37^{th} day post inoculation; however, animals inoculated with OCT4-negative cells began developing tumors on the 50^{th} day. The results also indicate that the isolated EGFP-positive cells expressed OCT4, EGFP, CD44, AC133 and ES cell marker SSEA4 as shown in Table 2.

TABLE 2
FACS analysis for stem cell markers in MDA-MB
231 breast cancer cells and control stem cells
positive (results are given in percentage)

Markers

| Cells | Oct3/4 | CD44 | AC133 | SSEA-4 |
|---|---|---|---|---|
| MB 231 | 89.67 | 97.02 | 20.04 | 64.80 |
| Control stem cells | 91.05 | 30.31 | 25.13 | 77.70 |

### EXAMPLE 6

### Fluorescence Biomarker Driven by the Oct3/4 Promoter is Strong and Specific for Cancer Stem Cells

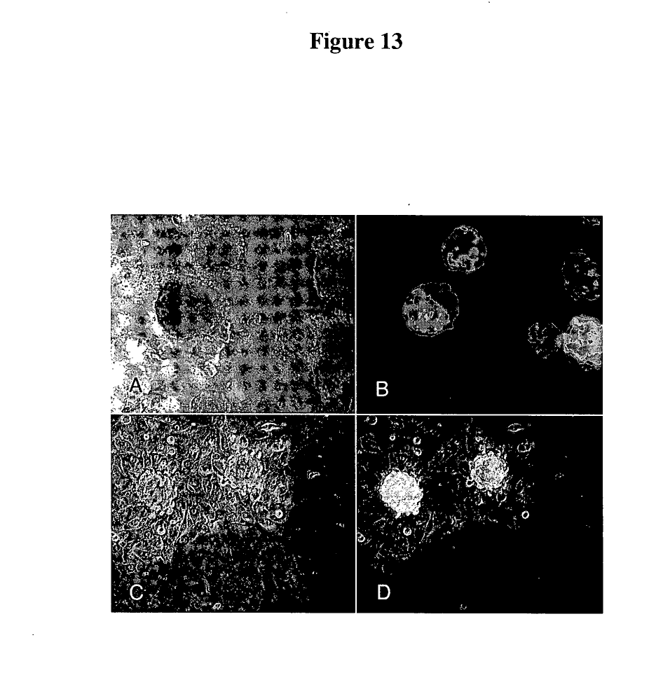

Mammosphere cultures were derived from an MDA-MB-435 melanoma cell line and biomarked for the presence of cancer stem cells expressing Oct-3/4. MDA-MB-435 cells were stably transfected with Oct4hP-eGFP and CMV-mRFP. The cells were then FACS sorted for GFP expressing cells to create a highly pure cancer stem cell population for further studies. Fluorescent micrograph of suspended tumor-derived spheres shown in FIG. 13B demonstrated that the fluorescence biomarker driven by the Oct3/4 promoter is strong and specific for cancer stem cells. Furthermore, fluorescent micrograph of the attached tumor spheres shown (FIG. 13D) exhibited a similar phenomenon. These findings demonstrate that fluorescence driven by the Oct3/4 promoter is strong and specific for cancer stem cells biomarked for study as therapeutic targets in various systems.

What is claimed is:

1. An isolated neoplastic stem cell population enriched for expression of OCT4.

2. The population of claim 1, wherein said population is characterized by OCT4^{hi} expression.

3. A composition comprising the population of claim 1.

4. The composition of claim 3, further comprising a cell culture media, having about 50% of said media derived from media cultures of primary human foreskin fibroblasts.

5. The composition of claim 4, wherein said media comprises a maximum of up to about 15% serum.

6. The composition of claim 4, wherein said media is depleted for EGF, FGF2, insulin, or combinations thereof.

7. A method of identifying a neoplastic stem cell, comprising the steps of:
(a) contacting a neoplastic cell population with an agent which specifically interacts with OCT4; and
(b) identifying a cell with which said agent specifically interacts;
thereby identifying a neoplastic stem cell.

8. The method of claim 7, wherein said agent comprises a nucleic acid.

9. The method of claim 7, wherein said agent is an OCT4 antibody.

10. The method of claims 7, wherein said identifying further comprises the use of a fluorescent microscope or fluorescence-activated cell sorter (FACS).

11. The method of claim 7, wherein said neoplastic cell is from a solid tumor.

12. The method of claim 7, wherein said neoplastic cell is metastatic.

13. A method for isolating a neoplastic stem cell, comprising the steps of:
(a) contacting a neoplastic cell population with an agent which specifically interacts with OCT4; and
(b) isolating a cell with which said agent specifically interacted thereby isolating a neoplastic stem cell.

14. The method of claim 13, wherein said agent comprises a nucleic acid.

15. The method of claim 13, wherein said agent is an OCT4 antibody

16. The method claim 13, wherein said neoplastic stem cell is isolated by fluorescence-activated cell sorter (FACS).

17. The method of claim 13, wherein said neoplastic cell is from a solid tumor.

18. The method of claim 13, wherein said neoplastic cell is metastatic.

19. A method of enriching a neoplastic cell population for neoplastic stem cells comprising the steps of:
(a) contacting a mixed cell population comprising a plurality of cancerous cells with a vector comprising an antibiotic resistance gene operatively linked to an Oct4 promoter; and
(b) culturing said cells in the presence of said antibiotic thereby enriching a neoplastic cell population for neoplastic stem cells.

20. The method of claim 19, wherein said culturing is conducted in a cell culture media, having 50% of said media derived from primary culture media of human foreskin fibroblasts.

21. The method of claim 19, wherein said mixed cell population is derived from a cell line.

22. The method of claim 19, wherein said mixed cell population is a primary cell culture derived from a tumor.

23. A method of inducing cancer comprising the step of:
introducing a neoplastic stem cell population enriched for expression of OCT4 to a mammal;
thereby inducing cancer.

24. The method of claim 23, wherein said neoplastic stem cell population initiate orthotopical tumor in-vivo.

25. The method of claim 24, wherein said neoplastic stem cell population initiate ectopical tumors in-vivo.

26. The method of claim 24, wherein said neoplastic stem cell population initiate metastases in-vivo.

27. A method of analyzing cancer progression and/or pathogenesis in-vivo comprising the steps of:
(a) transplanting OCT4^{hi} neoplastic stem cells into an animal; and
(b) analyzing cancer progression and/or pathogenesis in said animal;
thereby analyzing cancer progression and/or pathogenesis in-vivo.

28. The method of claim 27, further comprising the step of labeling said OCT4^{hi} neoplastic stem cells.

29. The method of claim 27, wherein, analyzing cancer progression comprises determining cell metastasis.

30. The method of claim 27, wherein, analyzing cancer progression comprises determining tumor progression.

31. The method of claim 27, further comprising the step of administering an agent of interest.

32. The method of claim 31, wherein, said agent is a therapeutic agent.

33. A method of treating, abrogating, or inhibiting cancer comprising the step of:
contacting a neoplastic cell with an agent that inhibits OCT4 expression or function in said cells;
thereby preventing, abrogating, or inhibiting cancer.

34. The method of claim 33, wherein said cancer comprises tumor growth.

35. The method of claim 33, wherein said cancer comprises cell metastasis.

36. The method of claim 33, wherein said agent comprises a nucleic acid.

37. The method of claim 33, wherein said agent is an OCT4 antibody.
